(19) United States
(12) Patent Application Publication
(10) Pub. No.: US 2023/0115360 A1
(43) Pub. Date: Apr. 13, 2023

(54) ENDOSCOPE, ACCESSORY, AND ENDOSCOPE SYSTEM

(71) Applicant: OLYMPUS MEDICAL SYSTEMS CORP., Tokyo (JP)

(72) Inventor: Kento IGUCHI, Tokyo (JP)

(73) Assignee: OLYMPUS MEDICAL SYSTEMS CORP., Tokyo (JP)

(21) Appl. No.: 17/958,975

(22) Filed: Oct. 3, 2022

Related U.S. Application Data
(60) Provisional application No. 63/253,275, filed on Oct. 7, 2021.

(51) Int. Cl. A61B 1/018; A61B 1/00; A61B 1/01

(52) U.S. Cl. CPC A61B 1/018; A61B 1/0008; A61B 1/01

(57) ABSTRACT

An endoscope according to an embodiment of the present disclosure includes an insertion portion configured to be inserted into a subject. A channel is provided in the insertion portion, the channel receiving an accessory. A plurality of first guides is spaced apart at intervals in the channel, the plurality of first guides configured to guide the accessory along a longitudinal axis of the insertion portion.

### RELATED APPLICATION DATA

This application is based on and claims priority under 37 U.S.C. § 119 to U.S. Provisional Application No. 63/253,275 filed on Oct. 7, 2021, the entire contents of which are incorporated herein by reference.

### FIELD OF THE DISCLOSURE

The present disclosure relates to an endoscope in which an accessory is inserted into a channel provided at an insertion portion, the accessory to be inserted into the channel provided at the insertion portion of the endoscope, and an endoscope system in which the accessory is inserted into the channel provided at the insertion portion.

### BACKGROUND

For example, a diagnosis of imaging a pancreatic duct, a bile duct, a hepatic duct, or the like, and treatment of collecting a gallstone in a common bile duct are performed using a treatment instrument inserted into a channel provided at an insertion portion of a so-called side-viewing endoscope.

The pancreatic duct is an extremely thin duct, and thus, it is not easy to insert the treatment instrument. Thus, after a distal end portion of the insertion portion is inserted near a duodenal papilla, a thin guide wire that is an accessory inserted into the channel is made to project in a side surface direction by a raising base (forceps elevator) of the distal end portion so as to be inserted into the pancreatic duct from a side opening of the distal end portion under X-ray fluoroscopy. Use of the guide wire as a guide makes it easy to insert the thick treatment instrument into the pancreatic duct. Further, insertion of the guide wire into the pancreatic duct allows the treatment instrument to be easily inserted and removed as many times as needed.

Japanese Patent Application Laid-Open Publication No. 2018-171255 discloses an endoscope capable of selecting a method for fixing a guide wire that is an accessory. In the endoscope, a center lock in which the guide wire is fixed at a first position of a raising base or a side lock in which the guide wire is fixed at a second position of the raising base more tightly than in a case of the center lock is selected. It is necessary to dispose the guide wire at the first position or the second position of the raising base to select a method for fixing the guide wire.

Japanese Patent No. 6138385 discloses an insertion support instrument having a bending shape for inserting an endoscope into an accessory nasal sinus. A distal end portion of the endoscope is fixed at the support instrument fitted in a guide of the insertion support instrument. As a result of the support instrument moving along the guide, the endoscope is inserted into the accessory nasal sinus along the bending shape of the insertion support instrument.

U.S. Patent Application Publication No. 2010/0268028 discloses a surgical device in which a treatment instrument that is an accessory is inserted into a subject using a guide provided on an inner wall of an insertion tube into which the treatment instrument is to be inserted.

### BRIEF SUMMARY OF EMBODIMENTS

An endoscope according to an embodiment of the present disclosure includes an insertion portion configured to be inserted into a subject. A channel is provided in the insertion portion, the channel receiving an accessory. A plurality of first guides is spaced apart at intervals in the channel, the plurality of first guides configured to guide the accessory along a longitudinal axis of the insertion portion.

An accessory according to another embodiment of the present disclosure includes a guided portion fitted with a plurality of first guides only at a distal end portion of the accessory. The accessory is inserted into a channel in an insertion portion configured to be inserted into a subject. The plurality of first guides is disposed in the channel and spaced apart at an interval.

An endoscope system according to another embodiment of the present disclosure is an endoscope system including an endoscope and an accessory. The endoscope includes an insertion portion configured to be inserted into a subject. A channel is provided in the insertion portion, the channel receiving the accessory. A plurality of first guides is configured to guide the accessory along a longitudinal axis of the insertion portion. The plurality of first guides is provided in the channel and spaced apart at an interval. The accessory includes a body configured to be inserted into the channel and a guided portion provided at the body and held in a state where the guided portion does not drop off from the plurality of first guides.

### BRIEF DESCRIPTION OF THE DRAWINGS

FIG. 1 is a perspective view of an endoscope system of a first embodiment.

FIG. 2 is a perspective view of a distal end portion of an endoscope of the first embodiment.

FIG. 3 is a cross-section diagram of the distal end portion of the endoscope of the first embodiment.

FIG. 4 is a top view of the distal end portion of the endoscope of the first embodiment.

FIG. 5 is a cross-section diagram along a line V-V in FIG. 4.

FIG. 6 is a schematic diagram of a channel of the endoscope of the first embodiment.

FIG. 7 is a schematic diagram for explaining a usage state of the endoscope of the first embodiment.

FIG. 8A is a schematic cross-section diagram of a bending portion of the endoscope of the first embodiment.

FIG. 8B is a schematic cross-section diagram of the bending portion of the endoscope of the first embodiment.

FIG. 9A is a cross-section diagram indicating a relationship between a guide and a guided portion.

FIG. 9B is a cross-section diagram indicating the relationship between the guide and the guided portion.

FIG. 9C is a cross-section diagram indicating the relationship between the guide and the guided portion.

FIG. 9D is a cross-section diagram indicating the relationship between the guide and the guided portion.

FIG. 9E is a cross-section diagram indicating the relationship between the guide and the guided portion.

FIG. 9F is a cross-section diagram indicating the relationship between the guide and the guided portion.

FIG. 10 is a schematic diagram for explaining a usage state of an endoscope of a modification 1 of the first embodiment.

FIG. 11 is a schematic diagram for explaining a usage state of an endoscope of a modification 2 of the first embodiment.

FIG. 12 is a view for explaining a guide of a second embodiment.

FIG. 13A is a view for explaining a guide of a modification of the second embodiment.

FIG. 13B is a view for explaining the guide of the modification of the second embodiment.

FIG. 13C is a view for explaining the guide of the modification of the second embodiment.

FIG. 14 is a top view of a distal end portion of an endoscope of a third embodiment.

### DETAILED DESCRIPTION

There is a possibility that a guide that guides an accessory, provided on an inner wall of an insertion tube may inhibit operability of an endoscope because drag is generated when the insertion tube is deformed. Further, as a result of the accessory rotating in the insertion tube, there is a possibility that it may be difficult to perform treatment.

An object of embodiments of the present disclosure is to provide an endoscope with favorable operability, an accessory of the endoscope with favorable operability, and an endoscope system with favorable operability.

According to the embodiments of the present disclosure, it is possible to provide an endoscope with favorable operability, an accessory of the endoscope with favorable operability, and an endoscope system with favorable operability.

### First Embodiment

Embodiments of the present disclosure will be described below with reference to the drawings. The drawings based on the embodiments are schematic diagrams, and a relationship between a thickness and a width of each portion, a ratio of a thickness, a relative angle, and the like, of each portion are different from the relationship and the ratio in an actual configuration. The drawings include portions where a relationship and a ratio of dimensions are different from each other. Illustration of part of components is omitted in the drawings.

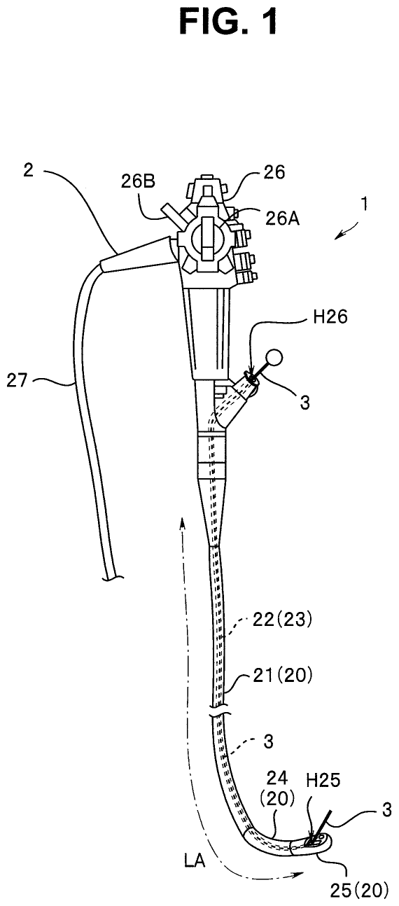

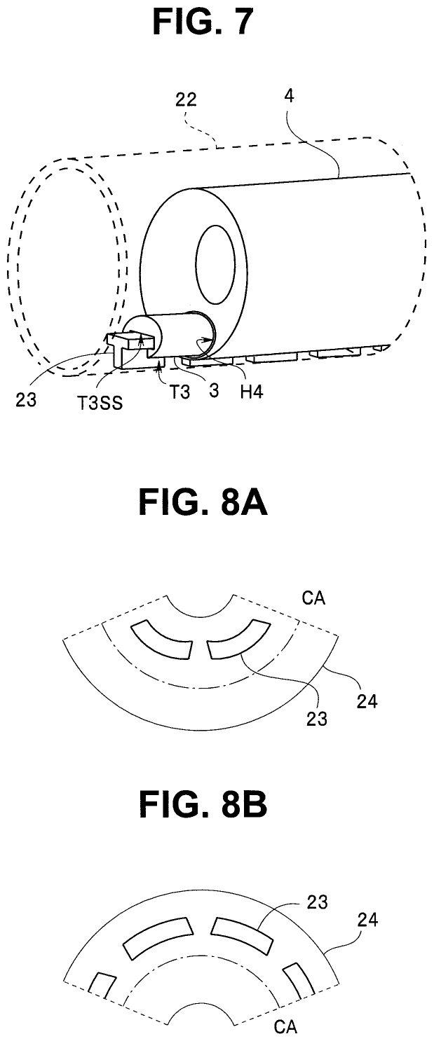

As illustrated in FIG. 1, an endoscope system 1 of the embodiment includes an endoscope 2, and a guide wire 3 that is an accessory. As will be described later, the guide wire 3 guides a treatment instrument 4 (see FIG. 7).

The treatment instrument 4 may be a so-called baby endoscope including an insertion portion having a smaller diameter than a diameter of the endoscope 2, as well as a catheter, a biopsy forceps, a snare, a high-frequency knife, a grasping forceps, a hemostatic clip, or the like.

The endoscope 2 includes an insertion portion (insertion tube) 20 to be inserted into a subject, an operation portion 26 to which a proximal end portion of the insertion portion 20 is connected, and a universal cord 27 extending from the operation portion 26. While not illustrated, the universal cord 27 is connected to a light source device and a video processor.

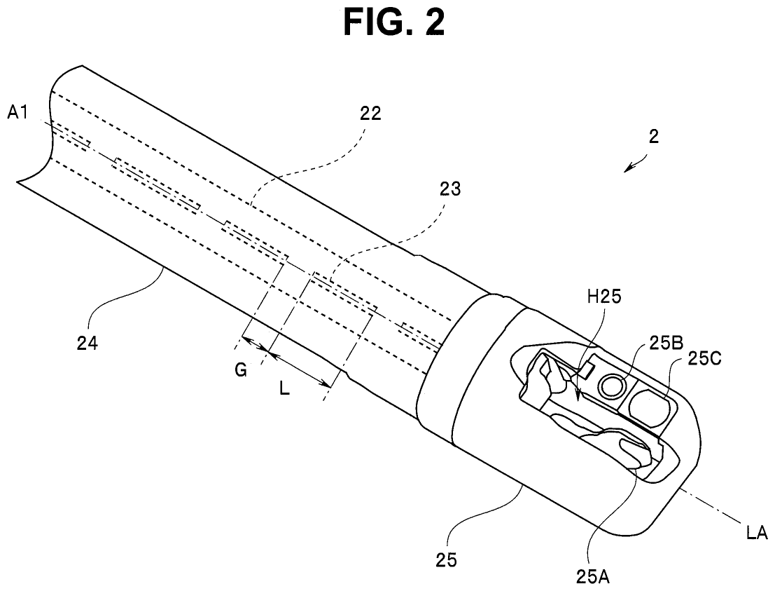

The insertion portion 20 that is elongated in a longitudinal axis direction LA includes a rigid distal end portion 25 at which a raising base 25A (see FIG. 2) which will be described later is disposed, a bending portion 24 continuously provided at a proximal end of the distal end portion 25, and a flexible tube 21 that is flexible and continuously provided at the proximal end of the bending portion 24. The bending portion 24 bends in accordance with operation of a bending operation knob 26A of the operation portion 26. A raising base operation knob 26B that raises up the raising base 25A is also provided at the operation portion 26.

The endoscope 2 includes a hollow elongated channel 22 that is an insertion tube into which the insertion portion is to be inserted from an insertion port H26 of the operation portion 26 to the distal end portion 25. The guide wire 40 that is an accessory of the endoscope 2 is inserted from the insertion port H26, passes through the hollow channel 22 and projects from a side opening H25 by raising up operation of the raising base 25A of the distal end portion 25.

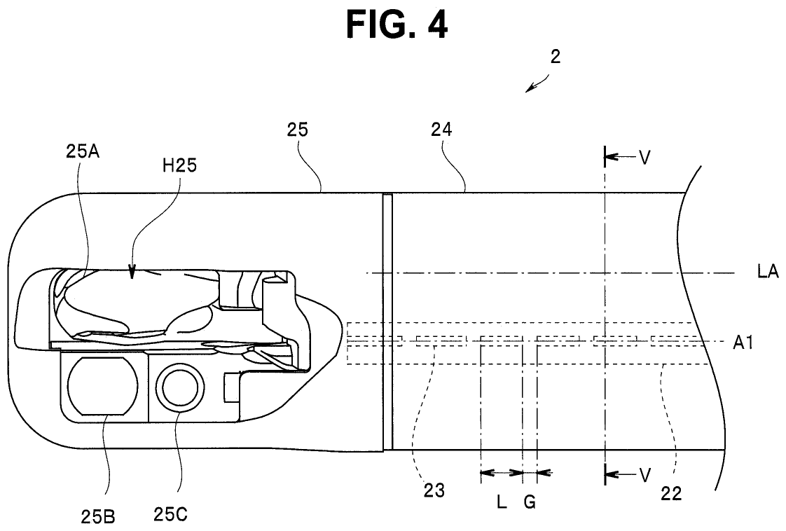

A configuration of the vicinity of the distal end portion 25 of the endoscope 2 will be described next using FIG. 2 to FIG. 4. The distal end portion 25 includes a rigid portion, for example, formed with a metal and a distal end cover formed with a non-conductive member such as a resin so as to cover a periphery of the rigid portion.

On a side surface of the distal end portion 25 of the endoscope 2 that is a side-viewing endoscope, an illumination window 25C that irradiates a side in the longitudinal axis direction LA with illumination light, and an image pickup optical system 25B that observes the side are disposed. The side opening H25 is an opening of space in which the raising base 25A is accommodated.

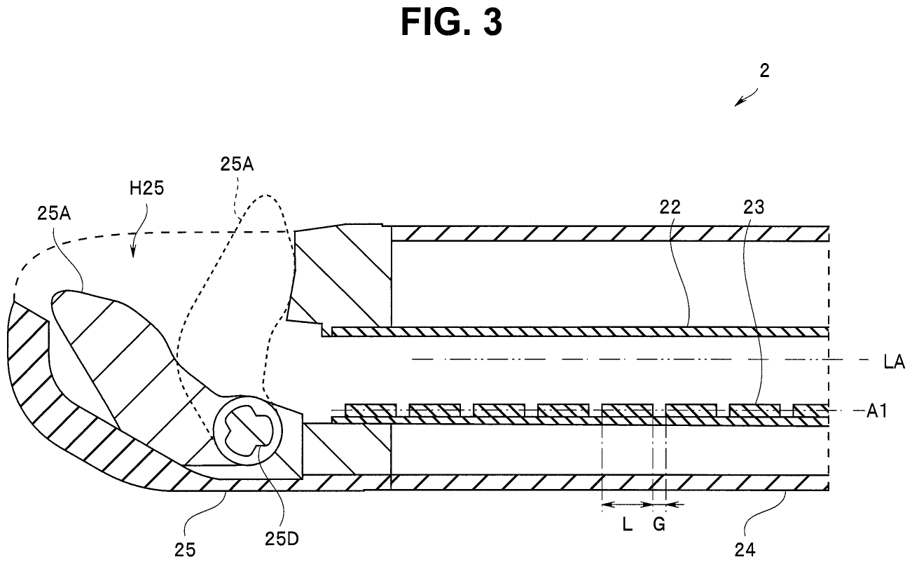

The raising base 25A guides the guide wire 3 that passes through the channel 22 and projects from the side opening H25 in a desired direction. The raising base 25A fixes the projecting guide wire 3 by holding the guide wire 3 with an outer edge of the side opening H25. As illustrated in FIG. 3, the raising base 25A rises up/falls down in accordance with rotation around a rotation axis 25D.

A core of the guide wire 3, formed with, for example, a super elastic alloy, is covered with a soft outer coat such as a fluorine resin and a urethane resin.

A distal end of the channel 22 is disposed at the distal end portion 25 and is open to face the raising base 25A. An opening (inner diameter) of the channel 22 is greater than an outer diameter of the guide wire 3. Thus, in the endoscope system 1 in related art, a position of the guide wire 3 projecting from the distal end of the channel 22 relative to the raising base 25A is not constant. The guide wire 3 is sometimes disposed at a central portion of the raising base 25A and sometimes disposed at an end portion. Further, the distal end of the guide wire 3 sometimes rotates in the channel 22.

A projecting direction and a rotation direction of the guide wire 3 are confirmed by an endoscope image acquired by the image pickup optical system 25B. In the endoscope system 1 in related art, it is necessary to adjust the distal end of the guide wire 3 to achieve a desired projecting direction and a desired rotation state by repeating rising up and falling down of the raising base 25A or operating the proximal end of the guide wire 3.

In contrast, a plurality of first guides 23 are disposed along the longitudinal axis LA of the insertion portion 20 in (on the inner wall of) the channel 22 of the endoscope 2 of the embodiment. In other words, the plurality of first guides 23 are located on a first axis A1 that is parallel to the longitudinal axis LA.

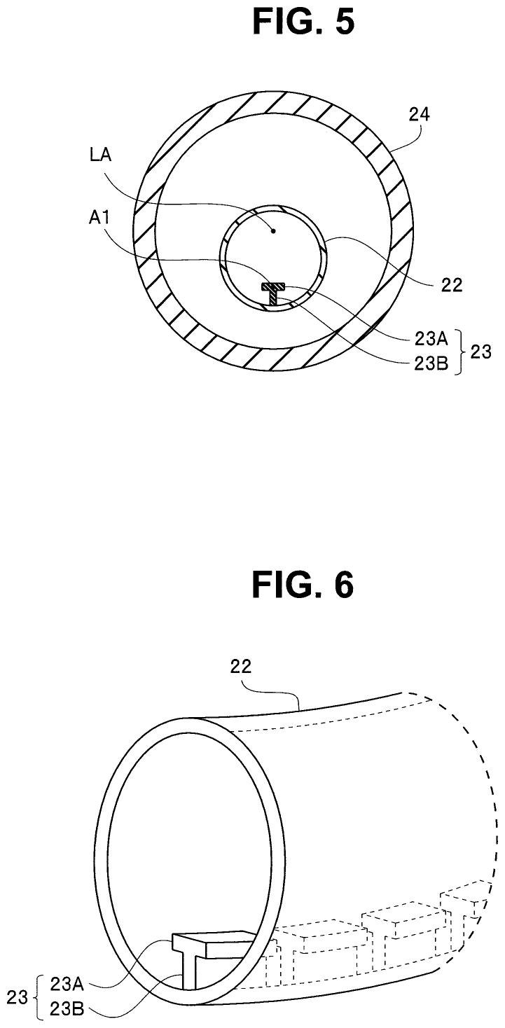

As illustrated in FIG. 5 and FIG. 6, the first guide 23 includes a rail 23A that is fitted with the guide wire 3, and a support portion 23B. As illustrated in FIG. 7, the guide wire 3 that is an accessory to be inserted into the channel 22 includes a trench T3 along the longitudinal axis direction LA. The trench T3 is a guided portion including an inner surface T3SS that is fitted with the first guide 23 provided at the body and is held in a state where the guided portion does not drop off from the first guide.

If the rail 23A has a width in a direction orthogonal to the longitudinal axis direction LA, wider than a width of the support portion 23B, a trench of the guide wire 3 fitted with the rail 23A does not drop off from the rail 23A. The guide wire 3 is guided to a predetermined position in a plane orthogonal to the longitudinal axis direction LA of the channel 22 by the plurality of first guides 23 and is disposed at a predetermined position of the raising base 25A in a predetermined rotation state.

To accurately dispose the guide wire 3 at the predetermined position of the raising base 25A, part of a distalmost first guide among the plurality of first guides 23 may be located in the distal end portion 25 of the insertion portion 20.

As illustrated in FIG. 7, the treatment instrument 4 includes a hole H4 that is a guided portion having a wall surface fitted with the guide wire 3. Thus, the treatment instrument 4 is guided to the predetermined position in a plane orthogonal to the longitudinal axis direction LA of the channel 22 by the guide wire 3.

If a guide provided on the inner wall of the channel 22 that is an insertion tube continues along the longitudinal axis of the channel 22, drag is generated when the insertion portion 20 (the flexible tube 21, the bending portion 24) is deformed, which may inhibit operability of the endoscope.

In contrast, in the endoscope 2 of the embodiment, the plurality of first guides 23 is spaced apart at predetermined intervals G. Thus, as illustrated in FIG. 8A and FIG. 8B, for example, when the bending portion 24 bends along a central axis CA, the first guide 23 does not generate drag with respect to bending operation.

The predetermined intervals G are, for example, beyond 1 mm or beyond 4.5 mm to prevent inhibition of deformation of the insertion portion 20. The predetermined intervals G may be less than 10 mm or less than 4.5 mm to prevent the guide wire 3 from dropping off from the first guide 23. Further, a length L in the longitudinal axis direction LA of the first guide 23 is, for example, less than 40 mm or less than 20 mm. The predetermined intervals G may be the same as the length L.

The predetermined intervals do not have to be constant. For example, a first interval of the plurality of first guides 23 in the bending portion 24 that are largely deformed particularly among the insertion portion 20 may be greater than a second interval of the plurality of first guides 23 in a portion other than the bending portion 24. Note that the plurality of first guides 23 may be spaced apart at the predetermined intervals G at least only in the bending portion 24 and may continue in a portion other than the bending portion 24.

The endoscope 2, which includes the first guides 23, can guide the guide wire 3 to a predetermined position of the distal end portion 25 in a predetermined rotation state. Further, the plurality of first guides 23 is spaced apart at the predetermined intervals G. Thus, deformation of the insertion portion 20 is not inhibited in the endoscope system 1 and the endoscope 2, so that favorable operability can be achieved.

A trench that is a guided portion of the guide wire 3 that is an accessory may be disposed in at least a distal end portion (distal end portion of the accessory) of the guide wire 3. A plurality of guided portions may be disposed. A length of the guided portion of the distal end portion of the guide wire is equal to or greater than 1.1 times of intervals G of the plurality of first guides 23 and may be equal to or greater than 1.24 times of the intervals G to prevent the guide wire 3 from dropping off from the first guide 23.

The guided portion of the guide wire 3 may be detachable from the distal end portion of the guide wire 3.

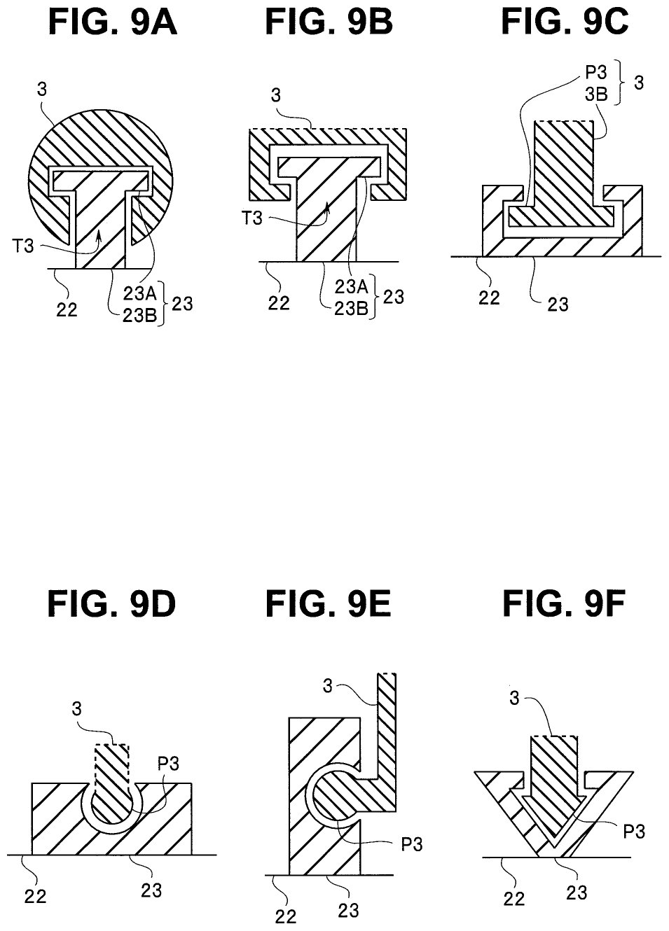

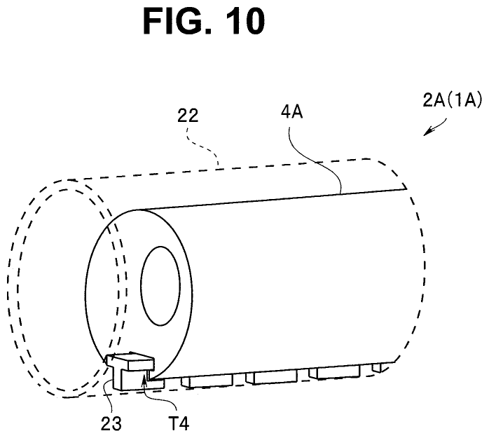

FIG. 9A to FIG. 9F illustrate combinations of the first guide 23 and the guide wire 3. The first guide 23 illustrated in FIG. 9A and FIG. 9B includes the rail 23A that is a guide, and a support portion 23B having a narrower width than a width of the rail 23A. The guide wire 3 includes a trench T3 that is a guided portion to be fitted with the rail 23A. The guide wire 3 illustrated in FIG. 9C includes a convex P3 that is a guided portion, and a support portion 3B having a narrower width than a width of the convex P3. The first guide 23 illustrated in FIG. 9D, FIG. 9E and FIG. 9F includes a trench that is an elongated concave, and the guide wire 3 includes a convex P3 that is a guided portion to be fitted with the trench of the first guide 23.

Combinations of the first guide 23 and the guide wire 3 are not limited to the illustrated configurations if the guided portion of the guide wire 3 does not drop off from the guide of the first guides 23 spaced apart at the predetermined intervals G.

For example, endoscopes 2A to 2D which will be described below are similar to the endoscope 2 and have the same effects. Thus, the same reference numerals will be assigned to components having the same functions as functions of the endoscope 2, and description will be omitted.

### Modification 1 of First Embodiment

In the endoscope 2A in the present modification illustrated in FIG. 10, the first guides 23 spaced apart at the predetermined intervals G guide a treatment instrument 4A including a trench T4 that is a guided portion to be fitted with a trench (hole) of the treatment instrument 4A. In other words, in the present disclosure, an accessory to be guided by the first guides 23 is the guide wire 3 or the treatment instrument 4A.

### Modification 2 of First Embodiment

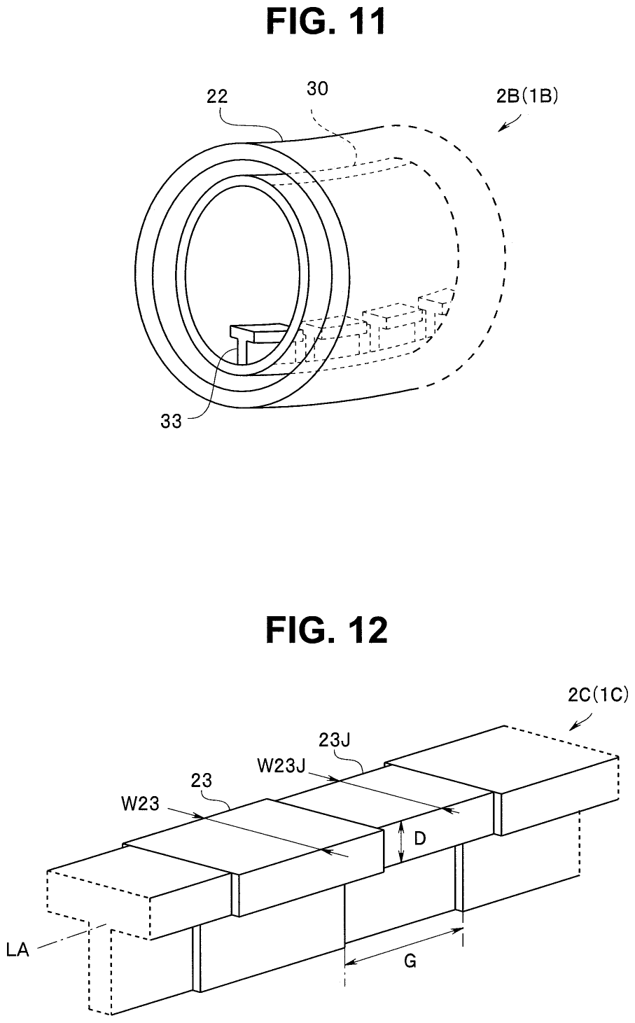

The endoscope 2B in the present modification illustrated in FIG. 11 further includes a flexible tube 30 in which a plurality of first guides 33 are disposed at predetermined intervals. The tube 30 is provided in the channel 22.

In the endoscope 2B, the tube 30 is inserted into the channel 22 for guiding an accessory. After the tube 30 is inserted into the channel 22, a position of the first guide 33 at a distal end of the tube 30 is adjusted and fixed at a position at the distal end of the channel at which the accessory is to be guided.

The endoscope 2B is not dedicated only for guiding an accessory, and thus, an accessory which is not required to be guided to a predetermined position can be easily inserted into the channel 22. Further, a position at which the accessory is to be guided can be adjusted, so that high versatility can be achieved.

### Second Embodiment

The endoscope 2C of the present embodiment illustrated in FIG. 12 includes a joint guide 23J that connects adjacent two first guides 23 and has a width W23J in a direction orthogonal to the longitudinal axis LA, narrower than the width W23 of the first guide 23. The joint guide 23J has a cross-section area in a direction orthogonal to the longitudinal axis LA, smaller than a cross-section area of the first guide 23 and is more likely to be deformed than the first guide 23, and thus, does not inhibit deformation of the insertion portion 20. Further, the first guide 23 including the joint guide 23J makes the accessory 3 less likely to drop off.

To prevent the joint guide 23J from inhibiting deformation of the insertion portion 20, for example, the width W23J is less than 80% or less than 50% of the width W23.

Further, a thickness D of a rail in a direction orthogonal to the longitudinal axis LA of the joint guide 23J may be thinner than a thickness of the rail of the first guide 23.

Note that in the endoscope including the bending portion, the bending portion is particularly largely deformed. Unlike with an endoscope including a bending portion that bends in four directions (upper, lower, right and left directions), in an endoscope including a bending portion that bends only in two directions (for example, upper and lower directions), at least one (cross-section area) of a width or a thickness in a bending direction (upper and lower direction) of the joint guide 23J is smaller than the width W23 of the first guide 23.

### Modification of Second Embodiment

An endoscope of the present modification has the same effects as the effects of the endoscope 2C of the second embodiment, and thus, the same reference numerals will be assigned to components having the same functions, and description will be omitted.

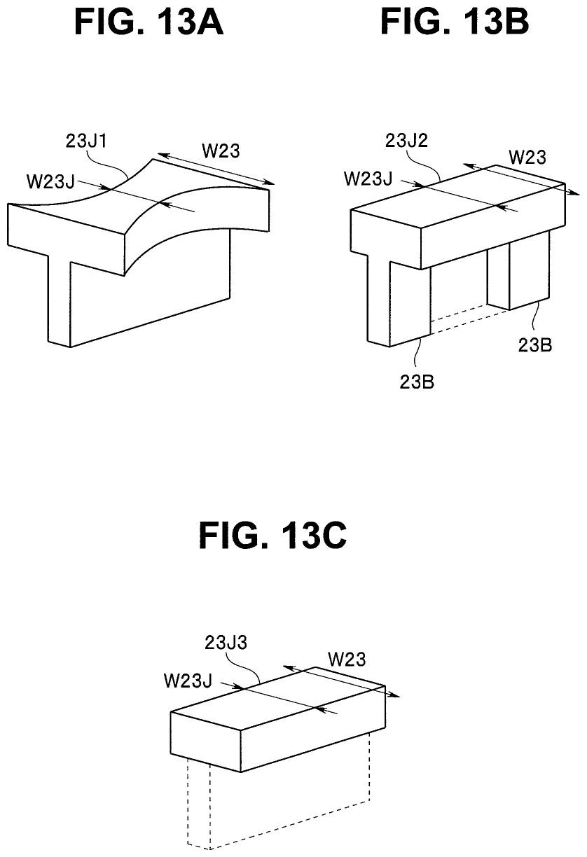

A joint guide 23J1 of the endoscope of the present modification illustrated in FIG. 13A includes a rail, a width W23J of which is not constant.

A joint guide 23J2 of the endoscope of the present modification illustrated in FIG. 13B includes a support portion 23B that is cut off.

A joint guide 23J3 of the endoscope of the present modification illustrated in FIG. 13C does not include a support portion 23B.

The joint guides 23J1, 23J2 and 23J3 have cross-section areas in a direction orthogonal to the longitudinal axis LA smaller than the cross-section area of the first guide 23, and thus, the accessory 3 does not drop off. The endoscope of the present modification provides the same effects as the effects of the endoscope 2 because the joint guides 23J1, 23J2 and 23J3 do not inhibit deformation of the insertion portion 20.

### Third Embodiment

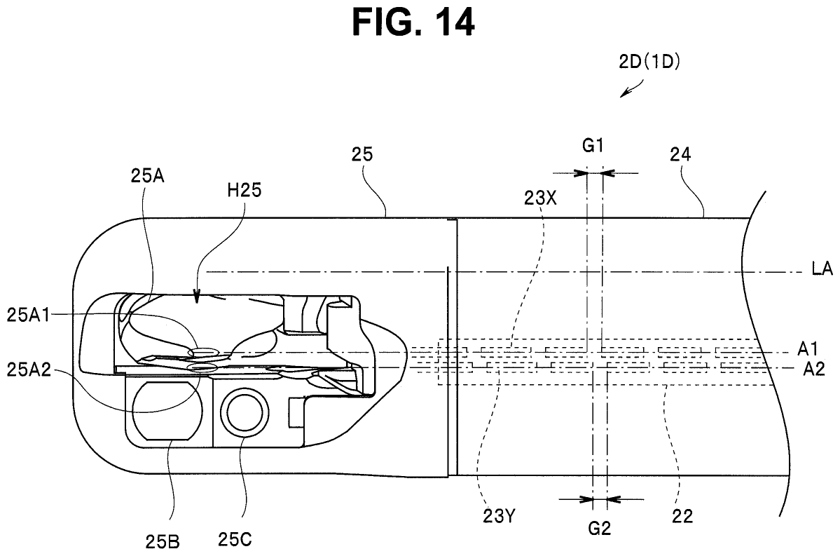

An endoscope 2D of the present embodiment illustrated in FIG. 14 includes a plurality of first guides 23X and a plurality of second guides 23Y in the channel 22. The plurality of first guides 23X is spaced apart at predetermined intervals G1 along a first axis A1. The plurality of second guides 23Y is spaced apart at predetermined intervals G2 along a second axis A2. The first axis A1 and the second axis A2 are parallel to the longitudinal axis LA of the insertion portion 20. The intervals G1 may be different from the intervals G2. Further, the first guides 23X and the second guides 23Y may have different shapes.

The raising base 25A includes a first fixing region 25A1 in which the guide wire 3 is held with an outer edge of the side opening H25 and center locked, and a second fixing region 25A2 in which the guide wire 3 is held with the outer edge of the side opening H25 and side locked.

The first fixing region 25A1 is located on the first axis A1, and the second fixing region 25A2 is located on the second axis A2.

The guide wire 3 inserted in a state where the guide wire 3 is guided by the first guide 23X at the insertion port H26 of the operation portion 26 is guided to the first fixing region 25A1 of the raising base 25A. Meanwhile, the guide wire 3 inserted in a state where the guide wire 3 is guided by the second guide 23Y is guided to the second fixing region 25A2 of the raising base 25A.

Operation of selecting center lock and side lock is easy, so that the endoscope 2D has favorable operability.

The endoscope may include a plurality of third guides spaced apart at predetermined intervals along the first axis A1 in addition to the plurality of first guides 23X and the plurality of second guides 23Y.

The endoscope system 1 and the endoscope 2 may be used in a medical field or an industrial field. It goes without saying that the endoscope system including the endoscopes 2A to 2D provides the effects of the endoscopes 2A to 2D in addition to the effects of the endoscope system 1.

The present disclosure is not limited to the above-described embodiments, and the like, and various changes, combinations and application are possible within a range not deviating from the gist of the disclosure.

What is claimed is:

1. An endoscope, comprising:
an insertion portion configured to be inserted into a subject;
a channel provided in the insertion portion, the channel receiving an accessory; and
a plurality of first guides spaced apart at intervals in the channel, the plurality of first guides configured to guide the accessory along a longitudinal axis of the insertion portion.

2. The endoscope according to claim 1, wherein the accessory is a guide wire that guides a treatment instrument.

3. The endoscope according to claim 1, wherein the accessory is a treatment instrument.

4. The endoscope according to claim 1, wherein the plurality of first guides includes a distalmost first guide, the distalmost first guide being located in a distal end portion of the insertion portion.

5. The endoscope according to claim 1, wherein the plurality of first guides is located on a first axis that is parallel to the longitudinal axis.

6. The endoscope according to claim 1, wherein the insertion portion includes a bending portion,
wherein the plurality of first guides is spaced apart at a first interval in the bending portion,
wherein the plurality of first guides is spaced apart at a second interval in a portion of the insertion portion other than the bending portion, and
wherein the first interval is greater than the second interval.

7. The endoscope according to claim 1, further comprising a tube in which the plurality of first guides is disposed, wherein the tube is provided in the channel.

8. The endoscope according to claim 1, further comprising a plurality of joint guides connecting adjacent two first guides of the plurality of first guides, wherein the plurality of joint guides has a width narrower than a width of the first guides.

9. The endoscope according to claim 8, wherein the plurality of joint guides has a thickness smaller than a thickness of the first guides.

10. The endoscope according to claim 5, further comprising a plurality of second guides spaced apart at intervals on a second axis parallel to the first axis in the channel, wherein the plurality of second guides is configured to guide the accessory along the longitudinal axis.

11. The endoscope according to claim 10, further comprising a raising base provided at a distal end portion of the insertion portion and configured to change a projecting direction of the accessory,
wherein the raising base includes:
a first fixing region in which the accessory is held with the distal end portion, and
a second fixing region in which the accessory is held with the distal end portion,
wherein the first fixing region is located on the first axis, and
wherein the second fixing region is located on the second axis.

12. The endoscope according to claim 11, comprising a rotation axis provided at the distal end portion, wherein the raising base is rotatably held on the rotation axis.

13. An accessory, comprising:
a guided portion fitted with a plurality of first guides only at a distal end portion of the accessory, the accessory inserted into a channel in an insertion portion configured to be inserted into a subject, the plurality of first guides disposed in the channel and spaced apart at an interval.

14. The accessory according to claim 13, wherein the guided portion has a length that is equal to or greater than 1.1 times the interval.

15. An endoscope system, comprising an endoscope and an accessory,
wherein the endoscope includes:
an insertion portion configured to be inserted into a subject,
a channel provided in the insertion portion, the channel receiving the accessory, and
a plurality of first guides configured to guide the accessory along a longitudinal axis of the insertion portion, and the plurality of first guides provided in the channel and spaced apart at an interval, and
wherein the accessory includes:
a body configured to be inserted into the channel, and
a guided portion provided at the body and held in a state where the guided portion does not drop off from the plurality of first guides.

16. The endoscope system according to claim 15, wherein the accessory is a guide wire that guides a treatment instrument.

17. The endoscope system according to claim 15, wherein the accessory is a treatment instrument.

18. The endoscope system according to claim 15, wherein the guided portion is disposed at a distal end portion of the accessory.

19. The endoscope according to claim 1, wherein, in a plane perpendicular to the longitudinal axis of the insertion portion, at least a portion of the plurality of first guides have a T-shape.

20. The endoscope according to claim 4, wherein, in a plane perpendicular to the longitudinal axis of the insertion portion, the distalmost first guide has a T-shape.
